(19) United States
(12) Patent Application Publication
(10) Pub. No.: US 2019/0255089 A1
(43) Pub. Date: Aug. 22, 2019

(54) OLIGONUCLEOTIDES FOR TREATMENT OF SARCOMA

(71) Applicant: MEMORIAL SLOAN KETTERING CANCER CENTER, New York, NY (US)

(72) Inventors: Lee Spraggon, New York, NY (US); Marc Ladanyi, New York, NY (US); Luca Cartegni, New York, NY (US)

(21) Appl. No.: 16/341,232

(22) PCT Filed: Oct. 11, 2017
(86) PCT No.: PCT/US17/56093

§ 371 Date: Apr. 11, 2019

Related U.S. Application Data
(60) Provisional application No. 62/406,566, filed on Oct. 11, 2016.
(60) Provisional application No. 62/406,566, filed on Oct. 11, 2016.

(51) Int. Cl. A61K 31/711; C12N 15/113; A61P 35/00; C12Q 1/6886

(52) U.S. Cl. CPC A61K 31/711; C12N 15/1135; A61K 9/0019; C12Q 1/6886; A61P 35/00

(57) ABSTRACT

The present invention relates to the treatment of sarcomas that are driven by oncogenic chimeric transcription factors, such as Ewing Sarcoma and Desmoplastic Small Round Cell Tumor (DSRCT). In some embodiments, the present invention provides oligonucleotide molecules that can redirect the pre-mRNA splicing of these oncogenic products, resulting in the production of truncated and transcriptionally inactive versions of these oncogenic transcription factors. The present invention also provides compositions comprising such oligonucleotide molecules, and methods of treatment involving administration of such oligonucleotide molecules.

### CROSS-REFERENCE TO RELATED APPLICATIONS

This application claims the benefit of priority of U.S. Provisional Patent Application No. 62/406,566 filed on Oct. 11, 2016, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 10, 2017, is named MSKCC_016_WO1_SL.txt and is 1,206 bytes in size.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

Ewing Sarcoma and Desmoplastic Small Round Cell Tumor (DSRCT) are aggressive sarcomas occurring primarily in children and adolescents. While intensive multi-modality therapy has improved survival in patients with localized disease, outcomes for patients with metastatic and relapsed/refractory disease remains unacceptably low. As such new therapeutic strategies are urgently required. Both Ewing Sarcoma and Desmoplastic Small Round Cell Tumor (DSRCT), as well as many other sarcomas, are driven chromosomal translocations that result in the generation of oncogenic chimeric transcription factors. However, transcription factors have traditionally been considered to be “un-druggable” targets. The development of new therapeutic strategies capable of targeting these oncogenic transcription factors could provide major advances in treatment.

### SUMMARY OF THE INVENTION

The present invention is based, in part, on certain discoveries that are described in more detail in the “Examples” section of this patent application and the Figures referenced therein. In particular, it has now been demonstrated that certain “splice switching oligonucleotides” (“SSOs”) can effectively target two different oncogenic chimeric transcription factors—one involved in the pathogenesis of Ewing Sarcoma and the other involved in the pathogenesis of DSRCT. The Ewing sarcoma breakpoint region 1(EWSR1; also known as EWS) represents one of the most commonly involved genes in sarcoma translocations. For example, an EWS-WT1 translocation is a known driver of DSRCT, and an EWS-FLI1 translocation is a known driver of Ewing Sarcoma. The present invention provides SSOs that can redirect the pre-mRNA splicing of such EWS fusion transcription factors, through either targeted exon skipping (for EWS-WT1) or activation of intronic polyadenylation (for EWS-FLI1). As described further in the Examples section of this patent disclosure, administration of an SSO referred to herein as “LSE9a” to cells having an EWS-WT1 translocation causes an in-frame deletion within the DNA binding domain of the oncogenic transcription factor, resulting in attenuation of its transcriptional activity, induction of apoptosis, reduced growth of DSRCT cell lines, and reduced DSRCT tumor growth in vivo. Similarly, as also described in the Examples section of this patent disclosure, administration of an SSO referred to herein as “SASA8” to cells having an EWS-FLI1 translocation results in activation of intronic polyadenylation by suppression of U1 snRNP binding to 5′ splice sites, leading to terminal truncation of the EWS-FLI1 pre-mRNA, removing the DNA binding motif encoded in the FLI1 fusion partner, resulting in in the attenuation of EWS-FLI1 transcriptional activity with a concurrent reduction in Ewing Sarcoma cell viability.

Building on these discoveries, the present invention provides various compositions and methods that may be useful for the treatment of sarcomas.

For example, in one embodiment the present invention provides isolated oligonucleotide molecules that comprise, or consist essentially of, or consist of, LSE9a (SEQ ID NO. 1)—as further described in the below Detailed Description. Similarly, in another embodiment the present invention provides isolated oligonucleotide molecules that are capable of hybridizing to the LSE9 target sequence SEQ ID NO. 3, and/or that are substantially complementary to the LSE9 target sequence SEQ ID NO. 3—as further described in the below Detailed Description. In some of such embodiments the isolated oligonucleotide molecules comprise naturally-occurring nucleotides. In other such embodiments the isolated oligonucleotide molecules comprise man-made modified nucleotides. The present invention also provides compositions that contain such isolated oligonucleotide molecules, such as pharmaceutical compositions. In other embodiments the present invention also provides methods of inhibiting the growth of DSRCT cells and/or of inducing apoptosis in DSRCT cells in vitro or in vivo (for example in an animal model), such methods comprising contacting DSRCT cells with an effective amount of one of such isolated oligonucleotide molecules or compositions. In other embodiments the present invention also provides methods of treating DSRCT in subjects in need thereof, such methods comprising administering to the subject an effective amount of one of such isolated oligonucleotide molecules or compositions. In some such embodiments the subject has a EWS-WT1 chromosomal translocation. In preferred embodiments the subject is a human.

Similarly, in another embodiment the present invention provides isolated oligonucleotide molecules that comprise, or consist essentially of, or consist of, SASA8 (SEQ ID NO. 2)—as further described in the below Detailed Description. Similarly, in another embodiment the present invention provides isolated oligonucleotide molecules that are capable of hybridizing to the SASA8 target sequence SEQ ID NO. 4, and/or that are substantially complementary to the SASA8 target sequence SEQ ID NO. 4—as further described in the below Detailed Description. In some of such embodiments the isolated oligonucleotide molecules comprise naturally-occurring nucleotides. In other such embodiments the isolated oligonucleotide molecules comprise man-made modified nucleotides. The present invention also provides compositions that contain such isolated oligonucleotide molecules, such as pharmaceutical compositions. In other embodiments the present invention also provides methods of inhibiting the growth of Ewing Sarcoma cells and/or of inducing apoptosis in Ewing Sarcoma cells in vitro or in vivo (for example in an animal model), such methods comprising contacting Ewing Sarcoma cells with an effective amount of one of such isolated oligonucleotide molecules or compositions. In other embodiments the present invention also provides methods of treating Ewing Sarcoma in subjects in need thereof, such methods comprising administering to the subjects an effective amount of one of such isolated oligonucleotide molecules or compositions. In some such embodiments the subject has a EWS-FLI1 chromosomal translocation. In preferred embodiments the subject is a human. While some embodiments of the present invention are summarized above, additional embodiments and aspects are described in the Brief Description of the Drawings/Figures, Detailed Description of the Invention, Examples, Claims, and Drawings/Figures sections of this patent application. The description in each section of this patent disclosure, regardless of any heading or sub-heading, is intended to be read in conjunction with all other sections of this patent disclosure. Furthermore, the various embodiments described in each section of this disclosure can be combined in various different ways, as will be apparent to those of ordinary skill in the art, and all such combinations are intended to fall within the scope of the present invention.

### BRIEF DESCRIPTION OF THE DRAWINGS

FIG. 1 provides a schematic representation of the approach used to generate “splice-switching oligonucleotides” (SSOs) to redirect the pre-mRNA splicing of the oncogenic EWS-WT1 fusion transcription factor through induction of exon skipping, as described in further detail in Example 1.

FIG. 2 provides the results of experiments showing that deletion of exon 9 resulted in the generation of a truncated ESW-WT1 protein product referred to as EWS-WT1ΔEx9 (see Western blot, lower left panel), and that in assays performed using a reporter gene comprising the promoter of the EWS-WT1 target gene ASCL1, driving expression of a luciferase reporter gene, deletion of exon 9 reduced the transcriptional activity of the EWS-WT1 transcription factor. The top left panel of FIG. 2 provides a schematic representation of the reporter gene experiment. The right panel of FIG. 2 provides data from such a reporter gene assay, showing relative luciferase activity (as a surrogate of ASCL1 promoter activity) in the presence of either full length EWS-WT1 or EWS-WTΔEx9.

FIG. 3 provides the results of RT-PCR experiments showing that the LSE9a SSO, but not a control SSO, induces a dose dependent switch in the EWS-WT1 mRNA product—generating a truncated mRNA product referred to as EWS-WT1ΔEx9.

FIG. 4A-B. FIG. 4A provides the results of experiments performed to validate certain patient-derived DSRCT cell lines. The top two panels of FIG. 4A provide RT-PCR (top left) and Western blot (top right) data obtained using the JN-DSRCT-1, BER-DSRCR-1 and BOD-DSRCT-1 cell lines. The lower panel of FIG. 4A provides fluorescent in situ hybridization (FISH) data obtained using the JN-DSRCT-1, BER-DSRCT-1 and BOD-DSRCT-1 cell lines. FIG. 4B provides the results of experiments performed to generate two pre-clinical mouse models for DSRCT: a metastasizing orthotopic xenograft mouse model created by intraperitoneal (IP) injection of DSRCT cell lines into mice and also the generation of conventional subcutaneous xenograft tumors. The left panel of FIG. 4B provides growth curves for subcutaneous xenograft tumors following injection of BER-DSRCT-1, BOD-DSRCT-1 and JN-DSRCT-1 cells. The right panel of FIG. 4B shows the locations and sizes of tumors in mice at various times after IP injection of BER-DSRCT-1 cells—as visualized using luciferase imaging. Growth of the primary tumor and distant metastases can be seen.

FIG. 5. The left-hand panels of FIG. 5 provide the results of RT-PCR (upper left) and Western blotting (lower left) experiments showing expression of the truncated EWS-WT1ΔEx9 mRNA and protein products, respectively, in DSRCT cell lines treated with LSE9a. The middle and right-hand panels of FIG. 5 provide the results of quantitative RT-PCR experiments showing that treatment of DSRCT cell lines with LSE9a SSO reduces the expression of two EWS-WT1 target genes—namely LRCC15 (middle panel) and ENT4 (right hand panel).

FIG. 6 provides the results of experiments showing that treatment of five different DSRCT cell lines (JN-DSRCT-1, SK1-DSRCT, SK2-DSRCT, BER-DSRCT, BOD-DSRCT) with LSE9a SSO results in the induction of apoptosis—as measured in terms of relative Caspase3/7 activity. The left bars in each case are controls and the right bars are SSO treated.

FIG. 7 provides the results of experiments showing that administration of LSE9a SSO to mice having DSRCT xenograft tumors (BER-DSRCT xenograft) significantly reduces tumor growth in vivo. The graph plots tumor volume in mm³ against time in days. Diamond symbols (upper line on graphs is data generated using the control SSO. Square symbols (lower line of graph) is data generated using the LSE9a SSO.

FIG. 8 provides a schematic representation of the approach used to generate SSOs to redirect the pre-mRNA splicing of the oncogenic EWS-FLI1 fusion transcription factor through activation of intronic polyadenylation (IPA), as described in further detail in Example 2.

FIG. 9 provides the results of RT-PCR experiments showing that the SASA8 SSO induces a dose dependent activation of the intronic polyadenylation signal (PAS) in intron 8 of EWS-FL1 resulting in the generation of a truncated EWS-FLI1 mRNA product referred to as EWS-FLIΔEx9).

FIG. 10 provides the results of quantitative RT-PCR (left panel) and Western blotting (right panel) experiments showing that treatment of Ewing Sarcoma cell lines with SASA8 SSO results in reduction of transcriptional activity of EWS-FLI1—as indicated by a reduction in the expression of the EWS/FLII target gene NR0B1 at the mRNA (left panel) and protein (right panel) level.

FIG. 11. The left panel of FIG. 11 provides the results of experiments showing that treatment of Ewing Sarcoma cell lines with SASA8 SSO results in a reduction of cell viability. The right panel of FIG. 11 provides RT-PCR data confirming the generation of the truncated EWS-FLI1ΔEx9 mRNA in the cells treated with SASA8.

FIG. 12. Photographic images of 3 cell culture dishes—each containing one of three different NIH3T3 cell lines. The cell lines had been grown in DMEM supplemented with 1% (v/v) fetal bovine serum for 2 weeks and then stained with crystal violet to visualize cells. The top image/dish contains NIH3T3 cells containing a pCDNA3.1 empty vector as a negative control. The middle image/dish contains NIH3T3 cells expressing a cDNA vector encoding a truncated EWSR1-WT1 construct lacking exon9 (EWSR1-WT1 ΔE9). The bottom image/dish contains NIH3T3 cells expressing a cDNA vector encoding a full length EWSR1-WT1 construct (EWSR1-WT1).

### DETAILED DESCRIPTION

The sub-headings provided below, and throughout this patent disclosure, are not intended to denote limitations of the various aspects or embodiments of the invention, which are to be understood by reference to the specification as a whole. For example, this Detailed Description is intended to read in conjunction with, and to expand upon, the description provided in the Summary of the Invention section of this application.

### Definitions

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range. Where a numeric term is preceded by “about,” the term includes the stated number and values ±10% of the stated number.

As used herein the term “active agent” refers to one or more of the oligonucleotide molecules described herein, including, but not limited to, LSE9 (SEQ ID NO. 1) and SASA8 (SEQ ID NO. 2), and/or any of the variants thereof described or contemplated herein. The terms “active agent” and “oligonucleotide molecule of the invention” are used interchangeably herein.

Various other terms are defined elsewhere in this patent disclosure, where used. Furthermore, terms that are not specifically defined herein may be more fully understood in the context in which the terms are used and/or by reference to the specification in its entirety. Where no explicit definition is provided all technical and scientific terms used herein have the meanings commonly understood by those of ordinary skill in the art to which this invention pertains.

### Oligonucleotides

TABLE 1
Oligonucleotide Sequences

| | | Sequence |
|---|---|---|
| | | ID |
| Name | Nucleotide Sequence | Number |
| LSE9a | AGTTTACGCACTTGTTTTACCTGTA | SEQ ID |
| | | NO. 1 |
| SASA8 | CAGGCCAAGGTAAACTCACCAGGGT | SEQ ID |
| | | NO. 2 |
| LSE9 target | TACAGGTAAAACAAGTGCGTAAACT | SEQ ID |
| sequence (the | | NO. 3 |
| reverse | | |
| complement | | |
| of LSE9) | | |
| SASA8 target | ACCCTGGTGAGTTTACCTTGGCCTG | SEQ ID |
| sequence (the | | NO. 4 |
| reverse | | |
| complement | | |
| of SASA8) | | |

In some embodiments the oligonucleotide molecules of the invention comprise, or consist of, or consist essentially of, LSE9a (SEQ ID NO. 1). Also falling within the scope of the present invention are oligonucleotide molecules that have substantially the same function as LSE9a (including, but not limited to, variants of LSE9a) in that they have one or more of the following properties:
(a) Ability to bind to/hybridize with the LSE9 target sequence (i.e. the reverse complement of LSE9—SEQ ID NO. 3);
(b) Ability to bind to/hybridize with a target sequence that overlaps with a portion of the LSE9 target sequence/SEQ ID NO. 3;
(c) Substantial complementarity to the LSE9 target sequence/SEQ ID NO. 3;
(d) Ability to induce exon skipping of EWS-WT1 exon 9 during transcription;
(e) Ability to induce production of a truncated EWS-WT1ΔEx9 mRNA;
(f) Ability to induce production of a truncated EWS-WT1ΔEx9 transcription factor protein;
(g) Ability to induce production of a truncated EWS-WT1 transcription factor protein that has reduced ability to bind to an EWS-WT1 target gene, as compared to a non-truncated/wild-type EWS-WT1 transcription factor protein;
(h) Ability to induce production of a truncated EWS-WT1 transcription factor protein that has reduced ability to activate transcription of an EWS-WT1 target gene, as compared to a non-truncated/wild-type EWS-WT1 transcription factor protein;
(i) Ability to induce production of a truncated EWS-WT1 transcription factor protein that has reduced ability to activate transcription of a reporter gene comprising promoter sequences from an EWS-WT1 target gene, as compared to a non-truncated/wild-type EWS-WT1 transcription factor protein;
(j) Ability to induce production of a truncated EWS-WT1 transcription factor protein that has reduced ability to activate transcription of a reporter gene comprising promoter sequences from the EWS-WT1 target gene ASCL1, LRCC15, or ENT4, as compared to a non-truncated/wild-type EWS-WT1 transcription factor protein;
(k) Ability to inhibit growth of DSRCT cells in vitro or in vivo;
(l) Ability to inhibit growth of DSRCT tumors in vivo.

In some embodiments, the oligonucleotide molecules of the invention comprise, or consist of, or consist essentially of, SASA8 (SEQ ID NO. 2). Also falling within the scope of the present invention are oligonucleotide molecules that have substantially the same function as SASA8 (including, but not limited to, variants of SASA8) in that they have one or more of the following properties:
(a) Ability to bind to/hybridize with the SASA8 target sequence (i.e. the reverse complement of SASA8—SEQ ID NO. 4);
(b) Ability to bind to/hybridize with a target sequence that overlaps with a portion of the SASA8 target sequence/SEQ ID NO. 4;
(c) Substantial complementarity to the SASA8 target sequence/SEQ ID NO. 4;
(d) Ability to induce exon skipping of EWS-FLI1 exon 9 during transcription;
(e) Ability to induce production of a truncated EWS-FLI1ΔEx9 mRNA;
(f) Ability to induce production a truncated EWS-FLI1ΔEx9 transcription factor protein;
(g) Ability to induce production of a truncated EWS-FLI1 transcription factor protein that has reduced ability to bind to an EWS-FLI1 target gene, as compared to a non-truncated/wild-type EWS-FLI1 transcription factor protein;
(h) Ability to induce production of a truncated EWS-FLI1 transcription factor protein that has reduced ability to activate transcription of an EWS-FLI1 target gene, as compared to a non-truncated/wild-type EWS-FLI1 transcription factor protein;
(i) Ability to induce production of a truncated EWS-FLI1 transcription factor protein that has reduced ability to activate transcription of a reporter gene comprising promoter sequences from an EWS-FLI1 target gene, as compared to a non-truncated/wild-type EWS-FLI1 transcription factor protein;
(j) Ability to induce production of a truncated EWS-FLI1 transcription factor protein that has reduced ability to activate transcription of a reporter gene comprising promoter sequences from the EWS-FLI1 target gene NR0B1, as compared to a non-truncated/wild-type EWS-FLI1 transcription factor protein;
(k) Ability to inhibit growth of Ewing Sarcoma cells in vitro or in vivo.
(l) Ability to inhibit growth of Ewing Sarcoma tumors in vivo.

Suitable assays for assessing each of the above properties are either provided in the Examples section of this patent application or are well known in the art.

In some embodiments the variant sequences have at least 50% or 55% or 60% or 65% or 70% or 75% or 80% or 85% or 90% or 95% or 98% or 99% identity with a specified sequence provided herein (e.g. SEQ ID NO. 1 or SEQ ID NO. 2).

In some embodiments the variant sequences bind to/hybridize with a specified sequence provided herein (e.g. SEQ ID NO. 3 or SEQ ID NO. 4) under high stringency conditions.

In some embodiments the oligonucleotide molecules of the invention (including those for which specific sequences are provided, and the contemplated variants thereof), the oligonucleotide molecules are about 20 nucleotides in length, or about 25 nucleotides in length, or about 30 nucleotides in length, or about 35 nucleotides in length, or about 40 nucleotides in length, or about 45 nucleotides in length, or about 50 nucleotides in length.

In some embodiments the oligonucleotide molecules of the invention (including those for which sequences specific sequences are provided, and the contemplated variants thereof) are 30 nucleotides or less in length, or 35 nucleotides or less in length, or 40 nucleotides or less in length, or 45 nucleotides or less in length, or 50 nucleotides or less in length.

All of the oligonucleotide molecules described herein, and the variants thereof, can be generated using any suitable means for generating or synthesizing oligonucleotide molecules that are known in the art.

Furthermore, all of the oligonucleotide molecules described herein, and the variants thereof, can be generated using any suitable nucleotide building blocks known in the art, whether those are naturally occurring nucleotide building blocks or modified/man-made building blocks. For example, in some embodiments the oligonucleotides of the present invention may comprise ribonucleotides or deoxyribonucleotides. In some embodiments the oligonucleotides of the present invention may comprise non-naturally occurring man-made nucleotides modified by manipulation of their chemical backbone or side chains, or by covalent or non-covalent attachment of other moieties, or by any other suitable means known in the art. Such modifications may serve to improve the stability of the resulting oligonucleotides, or the cellular or tissue uptake of the resulting oligonucleotides, or otherwise enhance the efficacy of the resulting oligonucleotides. For example, in some embodiments directed to therapeutic applications non-naturally occurring man-made nucleotides may be used. Examples of such non-naturally occurring man-made nucleotides include, but are not limited to, phosphorodiamidate morpholino oligonucleotide (PMO) nucleotides, peptide nucleic acid (PNA) nucleotides, locked nucleic acid (LNA) nucleotides, bridged nucleic acids (BNA) nucleotides, and the like. In some embodiments the oligonucleotide molecules described herein, and the variants thereof, comprise one or more naturally occurring nucleotide building blocks. In some embodiments the oligonucleotide molecules described herein, and the variants thereof, consist entirely of naturally occurring nucleotide building blocks. In some embodiments the oligonucleotide molecules described herein, and the variants thereof, comprise one or more non-naturally occurring man-made modified nucleotide building blocks. In some embodiments the oligonucleotide molecules described herein, and the variants thereof, consist entirely of non-naturally occurring man-made modified nucleotide building blocks.

Furthermore, in some embodiments the oligonucleotides of the present invention may be linked (e.g. covalently or non-covalently) to other molecules, including but not limited to polypeptides, carbohydrates, lipids, lipid-like molecules, ligands, or small molecules, for example to enhance their stability or uptake. For example, in some embodiments an oligonucleotide of the present invention may be linked (e.g. covalently or non-covalently) to a cell penetrating peptide (CPP). Many suitable CPPs are known in the art. For example, CPPs that can be used include, but are not limited to, those described in Guidotti et al. (“Cell-Penetrating Peptides: From Basic Research to Clinics;” Trends in Pharmacological Sciences; April 2017; Vol. 38, No. 4, p. 406-424), O'Donovan et al. (“Parallel Synthesis of Cell-Penetrating Peptide Conjugates of PMO Toward Exon Skipping Enhancement in Duchenne Muscular Dystrophy” Nucleic Acid Therapeutics; Volume 25, Number 1, 2015; DOI: 10.1089/nat.2014.0512), and U.S. Patent Application Publication No. 2014/0342992 A1 (including, but not limited to, the “9B2” peptide referred to therein)—the entire contents of each of which references/patent applications are hereby incorporated by reference.

Similarly, in some embodiments, the oligonucleotides of the present invention may be provided within delivery vehicles such as nanoparticles or any other suitable vehicle to facilitate or enhance delivery of the oligonucleotides to their desired target. For example, in some embodiments, the oligonucleotides of the invention may be provided in a vehicle useful for targeted delivery of active agents to a sarcoma (such as a DSRCT sarcoma or an Ewing Sarcoma), or to sarcoma cells.

### Methods of Treatment

Several of the embodiments of the present invention involve methods for the treatment of a sarcoma in a subject in need thereof. In some such embodiments the sarcoma is Ewing sarcoma. In some such embodiments the sarcoma is desmoplastic small round cell tumor (DSRCT). In some embodiments sarcoma is driven by, or associated with, expression of an oncogenic chimeric transcription factor. In some embodiments the sarcoma is an Ewing sarcoma driven by a EWS-FLI1 chromosomal translocation. In some embodiments the sarcoma is a DSRCT driven by a EWS-WT1 chromosomal translocation.

As used herein, the terms “treat,” “treating,” and “treatment,” refer to therapeutic measures that result in a detectable (and preferably statistically significant) improvement in one or more clinical indicators or symptoms of a sarcoma in a subject, such as one of the specific forms of sarcoma referenced herein. For example, such terms encompass either transiently or permanently improving, alleviating, abating, ameliorating, relieving, reducing, inhibiting, preventing, or slowing at least one clinical indicator or symptom, preventing additional clinical indicators or symptoms, reducing or slowing the progression of one or more clinical indicators or symptoms, causing regression of one or more clinical indicators or symptoms, and the like. For example, “treating” a sarcoma according to the present invention includes, but is not limited to, reducing the rate of growth of a sarcoma (or of sarcoma cells), halting the growth of a sarcoma (or of sarcoma cells), causing regression of a sarcoma (or of sarcoma cells), reducing the size of a sarcoma (for example as measured in terms of tumor volume or tumor mass), reducing the grade of a sarcoma, eliminating a sarcoma (or sarcoma cells), preventing, delaying, or slowing recurrence (rebound) of a sarcoma, improving symptoms associated with a sarcoma, increasing survival time for a sarcoma patient, inhibiting or reducing spreading of a sarcoma (e.g. metastases), and the like.

In some embodiments the methods of treatment described herein may be performed in combination with other methods of treatment useful for the treatment of sarcomas (such as Ewing Sarcoma or DSRCT), including, but not limited to, administration of other active agents (e.g. drugs), surgical methods (e.g. for tumor resection), radiation therapy methods, treatment with chemotherapeutic agents, radiation therapy, immunotherapy, adoptive cell transfer (ACT), or treatment with an or any other suitable method. Similarly, in certain embodiments the methods of treatment provided herein may be employed together with procedures used to monitor disease status/progression, such as biopsy methods and diagnostic methods (e.g. MRI methods or other imaging methods).

### Subjects

The terms “subject,” “individual,” and “patient”—which are used interchangeably herein, are intended to refer to any subject, preferably a mammalian subject, and more preferably still a human subject, for whom diagnosis, prognosis, or therapy is desired. Mammalian subjects include humans, domestic animals, farm animals, sports animals, and zoo animals including, e.g., humans, non-human primates, dogs, cats, mice, rats, guinea pigs, and the like.

In some embodiments the subject has, or is suspected of having, or is at risk of developing, a sarcoma, such as Ewing sarcoma or DSRCT.

In some embodiments the subject has, or is suspected of having, or is at risk of developing, a sarcoma that is driven by, or associated with, expression of an oncogenic chimeric transcription factor. For example, in some embodiments the subject has, or is suspected of having, or is at risk of developing, an Ewing sarcoma driven by a EWS-FLI1 chromosomal translocation. Similarly, in some embodiments the subject has, or is suspected of having, or is at risk of developing, a DSRCT driven by a EWS-WT1 chromosomal translocation.

### Administration Routes

The various different “active agents” provided herein can be administered to a subject via any suitable route, including by systemic administration or by local administration. “Systemic administration” means that the active agent is administered such that it enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous. “Local administration” means that a pharmaceutical composition is administered directly to where its action is desired (e.g., at or near the site of a sarcoma), for example via direct intratumoral injection. It is within the skill of one of ordinary skill in the art to select an appropriate route of administration taking into account the nature of the specific active agent being used and nature of the specific sarcoma to be treated.

### Effective Amounts

An “effective amount” of any active agent, composition, or pharmaceutical composition disclosed herein is an amount sufficient to sufficient to achieve, or contribute towards achieving, one or more outcomes described in the “treatment” definition above. An appropriate “effective” amount in any individual case may be determined using standard techniques known in the art, such as dose escalation studies, and may be determined taking into account such factors as the nature of the active agent, the desired route of administration, the desired frequency of dosing, the specific sarcoma being treated, the subjects, age, sex, and/or weight, etc. Furthermore, an “effective amount” may be determined in the context of any other treatment to be used. For example, in those situations where an active agent as described herein is to be administered or used in conjunction with other treatment methods or other agents, then the effective amount may be less than it would be where no such additional treatment method is used.

### Methods for Determining Whether Subjects are Candidates for Treatment

In some embodiments the present invention involves determining whether a subject is a candidate for treatment using any of the compositions or methods provided herein. In some embodiments this involves determining or measuring or detecting the presence of an oncogenic chimeric transcription factor protein (e.g. one of the specific oncogenic chimeric transcription factors described herein), or a chromosomal translocation (e.g. one of the specific chromosomal translocations described herein) that would result in the expression of an oncogenic chimeric transcription factor protein, in a subject, such as in a sarcoma or sarcoma cell of the subject, whereby if the subject (or the subject's sarcoma or sarcoma cells) expresses the oncogenic chimeric transcription factor protein, or has a chromosomal translocation that would result in the expression of such an oncogenic chimeric transcription factor, then the subject may be a candidate for treatment.

### Compositions

Several of the embodiments of the present invention involve compositions, for example pharmaceutical compositions. The term “composition” refers to a composition comprising at least one of the “active agents” (i.e. oligonucleotide molecules) described herein, and one or more additional components—such as diluents, buffers, saline (such as phosphate buffered saline), cell culture media, and the like. Where such “compositions” are “pharmaceutical compositions” the one or more additional components must be components that are suitable for delivery to a living subject, such as diluents, buffers, saline (such as phosphate buffered saline), carriers, stabilizers, dispersing agents, suspending agents, and the like.

The term “pharmaceutical composition” refers to a preparation that is in such form as to permit the biological activity of the active agent to be effective, and which contains no additional components that are unacceptably toxic to a subject to which the composition would be administered. Pharmaceutical compositions can be in numerous dosage forms, for example, tablet, capsule, liquid, solution, soft-gel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

### EXAMPLES

The Ewing sarcoma breakpoint region 1 (EWSR1; also known as EWS) represents one of the most commonly involved genes in sarcoma translocations. This gene has a large number of fusion partners, driving the pathogenesis of soft tissue tumors. Targeting these oncogenic chimeric transcription factors could provide an effective therapy for the treatment of these sarcomas. However, this has proven elusive with convention small molecule approaches, indicating that other methods of inhibiting these oncogenic fusions are required. The present Examples, which are exemplary only and not intended to be limiting, provide such new methods.

### Example 1

### Desmoplastic Small Round Cell Tumor (DSRCT)

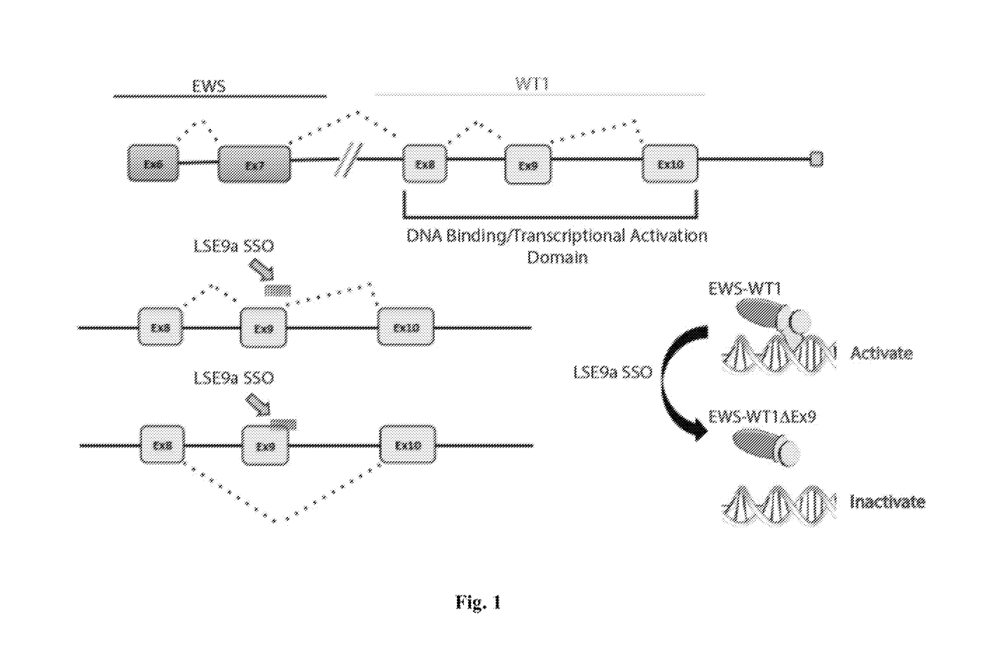

Splice-switching oligonucleotides (SSOs) were developed with the aim of redirecting the pre-mRNA splicing of EWS-WT1 fusion transcription factors through induction of exon skipping. FIG. 1 provides a schematic representation of the approach used—which involved designing an oligonucleotide that would bind to exon 9 of WT1 such that it could induce skipping of exon 9, thereby resulting in an in-frame deletion leading to generation of a mutant transcription factor protein lacking a portion of the DNA binding and transcriptional activation domain (which is encoded by exons 8, 9 & 10). A specific SSO referred to herein as “LSE9a” (having the sequence of SEQ ID NO. 1) was designed and produced—using phosphorodiamidate morpholino nucleotides. However, it should be noted that different oligonucleotide sequences could also be used. For example, variants of SEQ ID NO. 1 could be used, or other oligonucleotides that can cause skipping of exon 9 could be used, or indeed other oligonucleotides that can cause skipping of exon 8 or exon 10 could be used. Indeed any other oligonucleotides capable of binding to (hybridizing with) the LSE9a target sequence (i.e. the sequence complementary to LSE9a to which the LSE9a oligonucleotide binds), and/or capable of causing exon skipping that result in generation of a mutant transcription factor that is incapable of DNA binding and/or transcriptional activation could also be used. Furthermore, other types of modified or unmodified nucleotide building blocks could be used in place of phosphorodiamidate morpholino nucleotides.

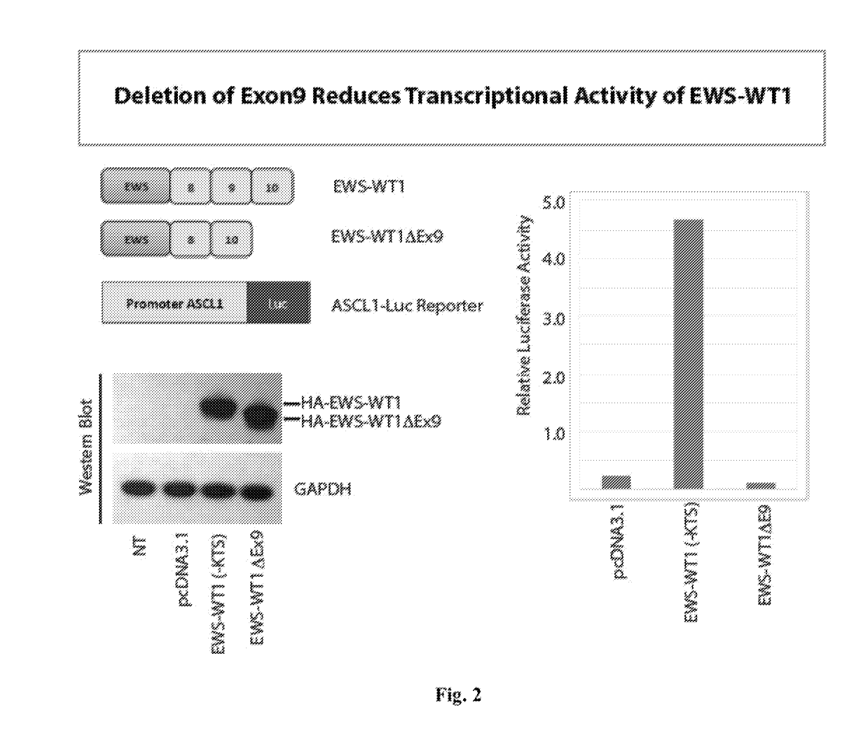

FIG. 2 provides the results of experiments showing that deletion of exon 9 resulted in the generation of a truncated ESW-WT1 protein product (EWS-WT1ΔEx9, the HA-tagged version thereof, see lower left panel) that had reduced transcriptional activity (see right hand panel). In particular, the truncated EWS-WT1ΔEx9 product was tested for its effect on a reporter gene comprising the promoter of the EWS-WT1 target gene ASCL1—driving expression of a luciferase reporter gene. The top left panel of FIG. 2 provides a schematic representation of the reporter gene experiment. The right panel of FIG. 2 provides data from such a reporter gene assay, showing relative luciferase activity (as a surrogate of ASCL1 promoter activity) in the presence of either full length EWS-WT1 or EWS-WTΔEx9—and showing that deletion of exon 9 reduced the transcriptional activity of the EWS-WT1 transcription factor.

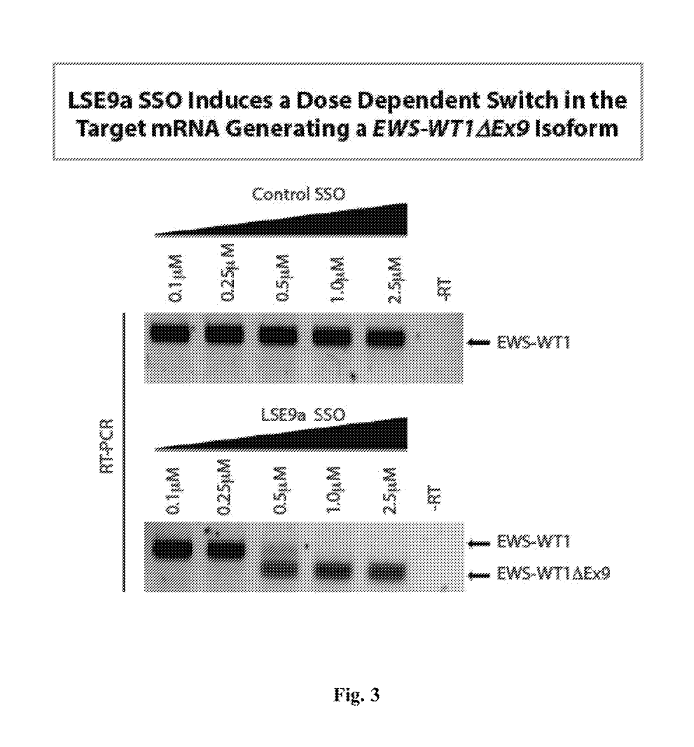

FIG. 3 provides the results of RT-PCR experiments showing that the LSE9a SSO induces a dose dependent switch in the EWS-WT1 mRNA product—generating a truncated mRNA product referred to as EWS-WT1ΔEx9.

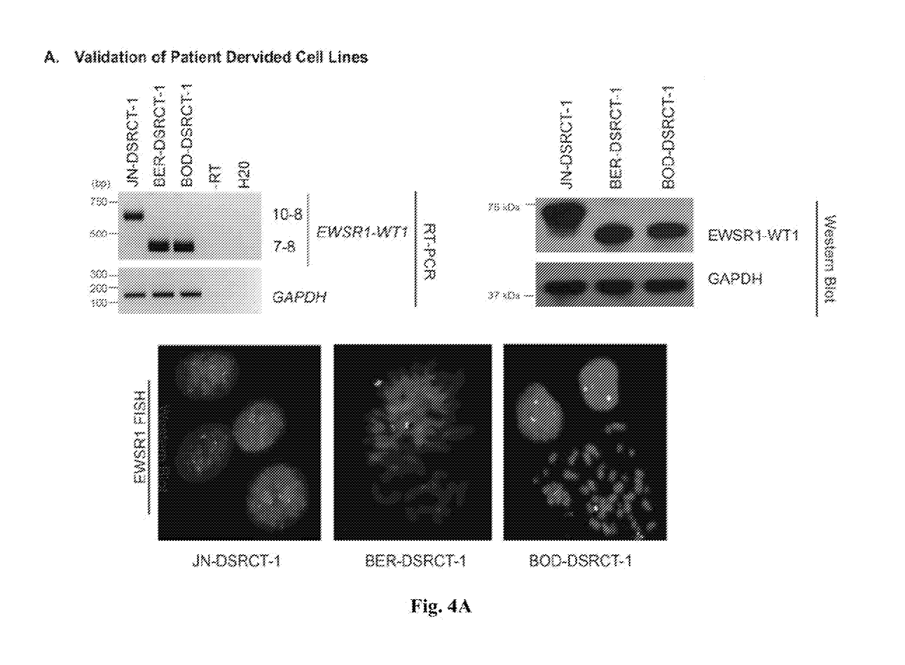

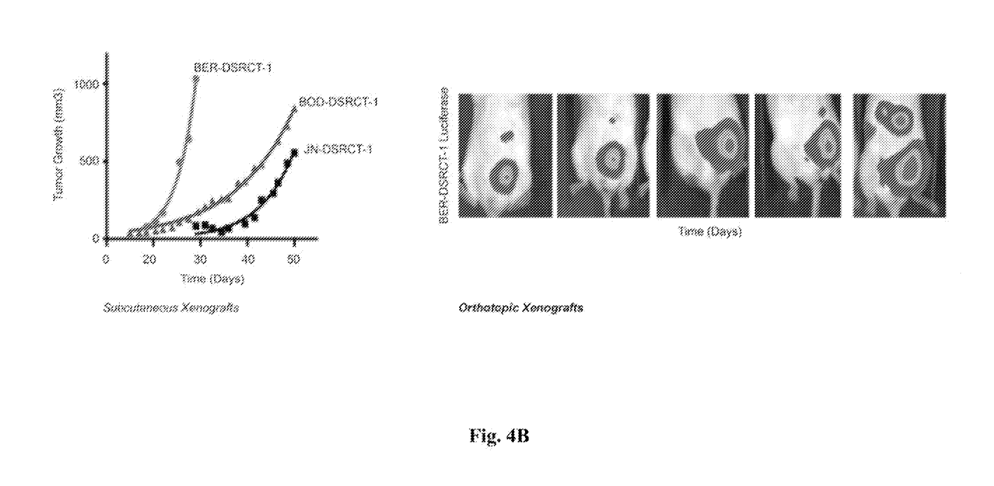

Two new DSRCT cell lines harboring the characteristic oncogenic EWSR1-WT1 t(11;22)(p13;q12) translocation/fusion were generated. Other publicly available DSRCT cell lines were also used in certain experiments (e.g. the publicly available JN-DSRCT-1 cell line). FIG. 4A provides the results of certain experiments performed to validate these cell lines. The top two panels of FIG. 4A provide RT-PCR and Western blot data showing that the BER-DSRCT-1 and BOD-DSRCT-1 cell lines express the EWS-WT1 fusion mRNA and protein. The lower panels of FIG. 4A provide images from fluorescent in situ hybridization (FISH) experiments showing that both cell lines contain the EWS-WT1 t(11;22)(p13;q12) fusion at the genomic level.

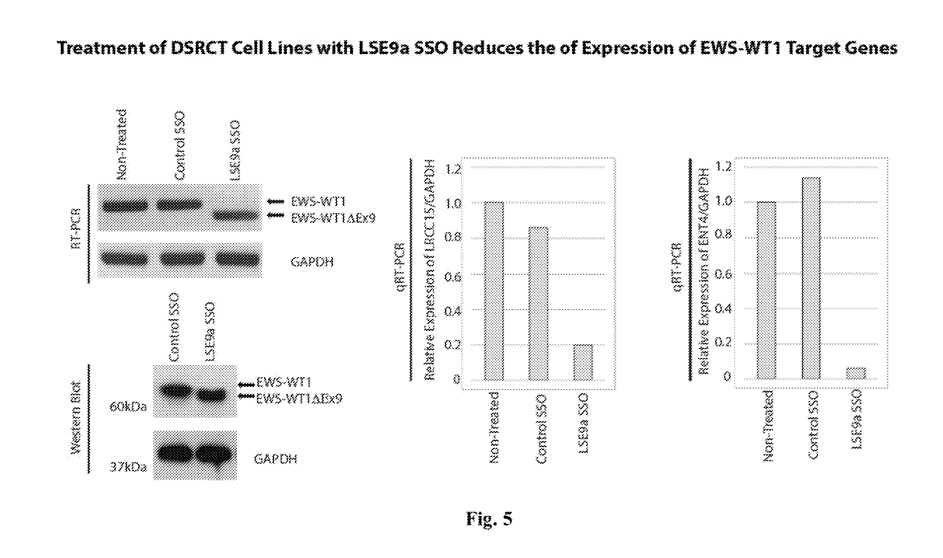

The left hand panels of FIG. 5 provide the results of RT-PCR and Western blotting experiments showing expression of the truncated EWS-WT1ΔEx9 mRNA and protein products, respectively, in DSRCT cell lines treated with LSE9a. The right and middle panels of FIG. 5 provide the results of quantitative RT-PCR experiments showing that treatment of DSRCT cell lines with LSE9a SSO reduces the expression of two EWS-WT1 target genes—namely LRCC15 (middle panel) and ENT4 (right panel).

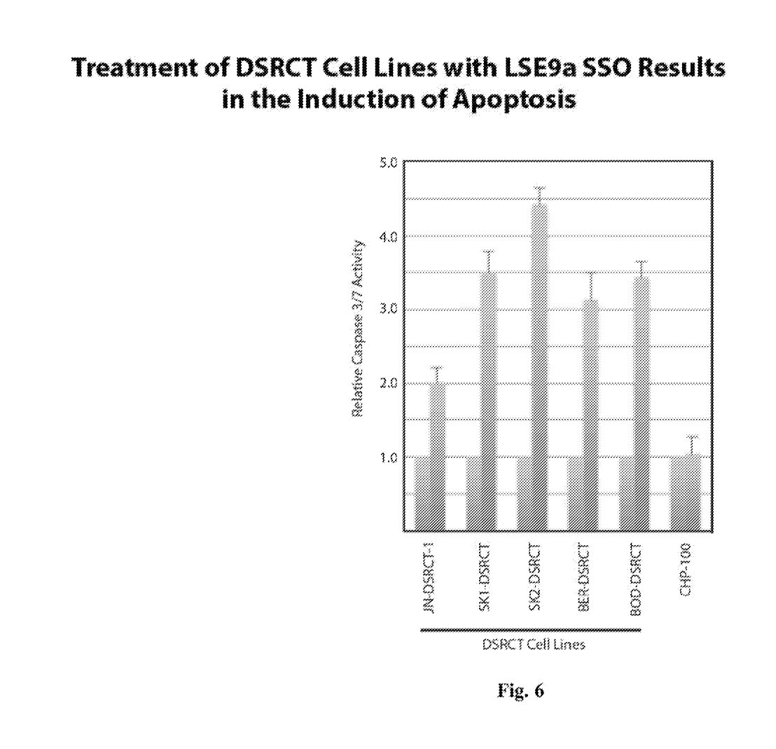

FIG. 6 provides the results of experiments showing that treatment of five different DSRCT cell lines (JN-DSRCT-1, SK1-DSRCT, SK2-DSRCT, BER-DSRCT, BOD-DSRCT) with LSE9a SSO results in the induction of apoptosis—as measured in terms of relative Caspase3/7 activity. The left (light gray) bars in each case are controls and the right (darker gray) bars are SSO treated.

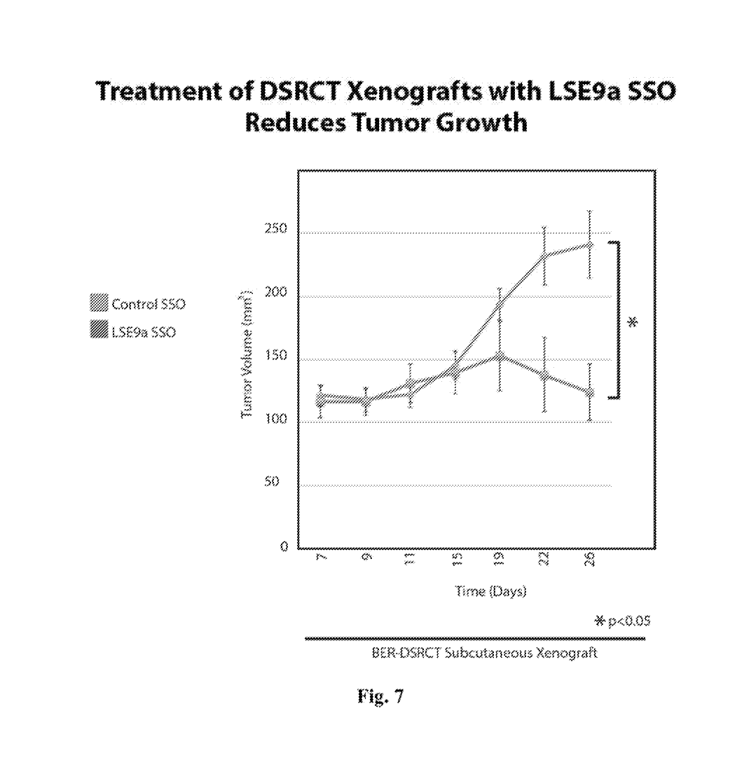

Some of the DSRCT cell lines described above were also used to develop a standard subcutaneous xenograft model and also metastasizing orthotopic xenograft mouse model. FIG. 4B provides the results of certain experiments performed to validate this model. The left panel of FIG. 4B provides growth curves for subcutaneous xenograft tumors following injection of BER-DSRCT-1, BOD-DSRCT-1 and JN-DSRCT-1 cell lines. The right panel of FIG. 4B shows the locations and sizes of tumors in the mice at various times after injection of BER-DSRCT-1 cells—as visualized using luciferase imaging. Growth of the primary tumor and distant metastases can be seen. FIG. 7 provides the results of experiments showing that administration of LSE9a SSO to mice having DSRCT xenograft tumors (BER-DSRCT xenograft) significantly reduces tumor growth in vivo.

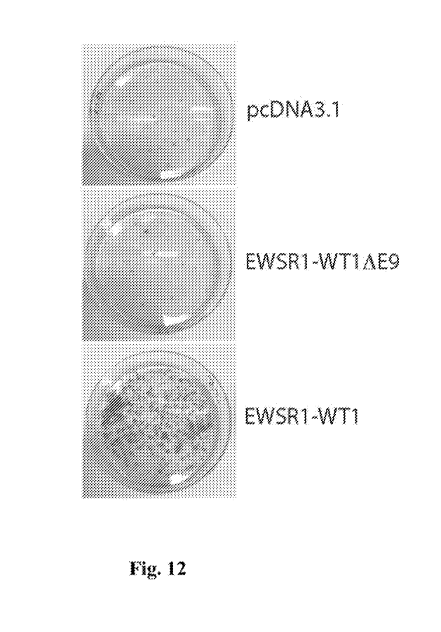

It was found that while expression of full length EWSR1-WT1 cDNA transforms NIH-3T3 cells, expression of an EWSR1-WT1 cDNA lacking exon 9 does not. FIG. 12. provides the results from these experiments. NIH3T3 cell lines expressing cDNA vectors for full length and truncated EWSR1-WT1 (lacking exon9) were generated, along with NIH3T3 cell lines containing pCDNA3.1, as negative control (empty vector). Cell lines were then analyzed for their dependency on serum. The growth of the three NIH3T3 cell lines in DMEM supplemented with 1% (v/v) in fetal bovine serum was assayed for 2 weeks and then stained with crystal violet to visualize cells—as shown in FIG. 12. NIH3T3 cells containing EWSR1-WT1 full length cDNA demonstrated significant growth potential in low serum, whilst NIH3T3 cells containing the EWSR1-WT1 fusion lacking the exon9 of WT1, had a significantly reduced growth potential comparable to that of NIH3T3 expressing the empty vector (pCDNA3.1). This confirms the reduced oncogenic potential of EWSR1-WT1 lacking exon 9.

### Example 2

### Ewing Sarcoma

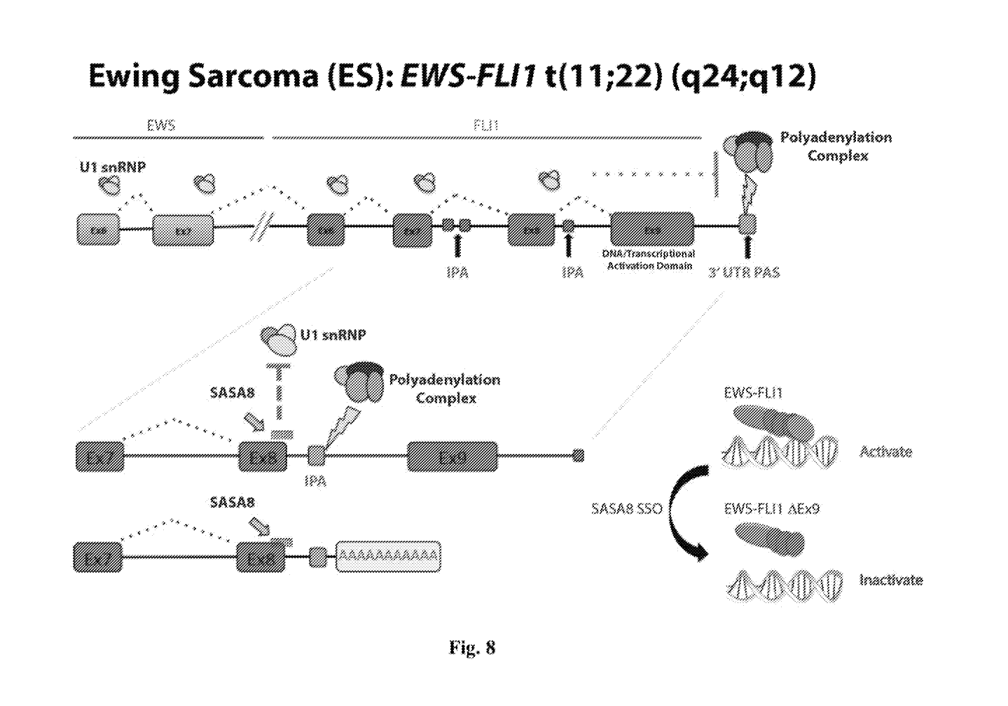

Splice-switching oligonucleotides (SSO) were developed with the aim of redirecting the pre-mRNA splicing of EWS-FLI1 fusion transcription factors through activation of intronic polyadenylation (IPA). FIG. 8 provides a schematic representation of the approach used—which involved designing an oligonucleotide that would bind to exon 8 of FLI1, resulting in activation of intronic polyadenylation by suppression of U1 snRNP binding to 5′ splice sites, resulting in terminal truncation of the EWS-FLI1 pre-mRNA to remove the DNA binding motif encoded in the FLI1 fusion partner. A specific SSO referred to herein as “SASA8” (having the sequence of SEQ ID NO. 2) was designed and produced using phosphorodiamidate morpholino nucleotides were used. (However, it should be noted that different oligonucleotide sequences could also be used. For example, variants of SEQ ID NO. 2 capable of binding to (hybridizing with) the SASA8 target sequence (i.e. the sequence complementary to SASA8 to which the SASA8 oligonucleotide binds) could be used, or other oligonucleotides that can cause activation of intronic polyadenylation, e.g. by suppression of U1 snRNP binding to 5′ splice sites, resulting in terminal truncation of the EWS-FLI1 pre-mRNA to remove or inactivate the DNA binding motif encoded in the FLI1 fusion partner, could be used. Furthermore, other types of modified or unmodified nucleotide building blocks could be used in place of phosphorodiamidate morpholino nucleotides.)

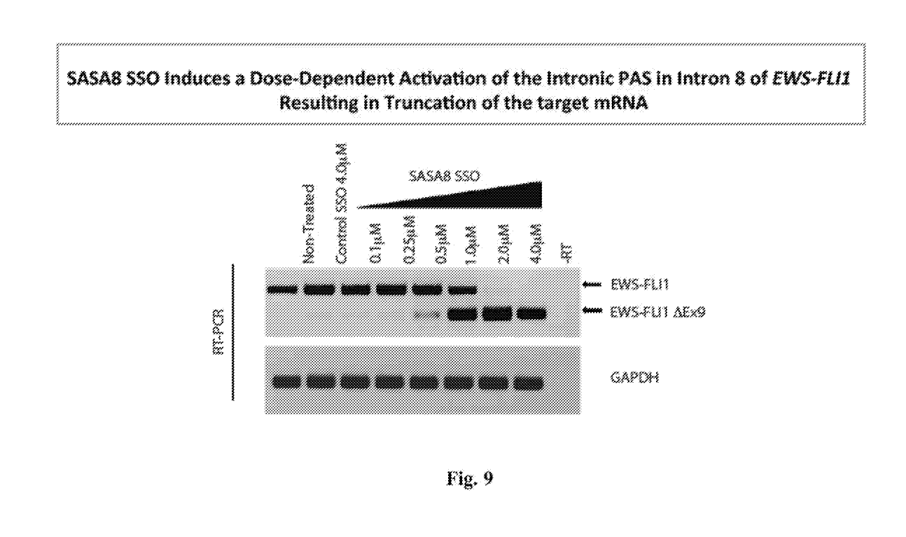

FIG. 9 provides the results of RT-PCR experiments showing that the SASA8 SSO induces a dose dependent activation of the intronic polyadenylation signal (PAS) in intron 8 of EWS-FLI1 resulting in the generation of a truncated EWS-FLI1mRNA product referred to as EWS-FLI1ΔEx9).

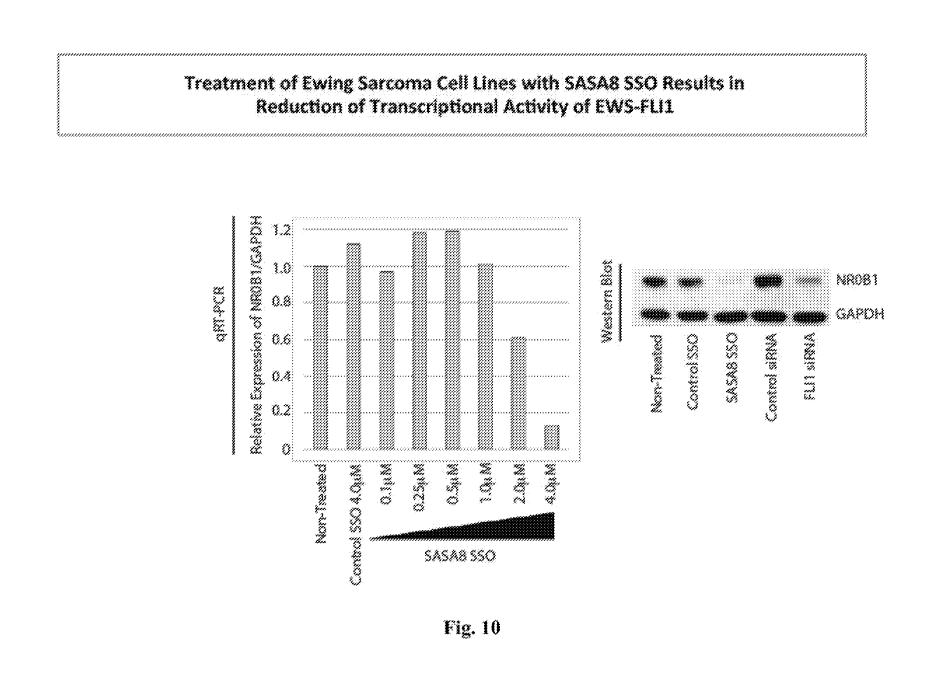

FIG. 10 provides the results of quantitative RT-PCR (left panel) and Western blotting (right panel) experiments showing that treatment of Ewing Sarcoma cell lines with SASA8 SSO results in reduction of transcriptional activity of EWS-FLI1—as indicated by a reduction in the expression of the EWS/FLII target gene NR0B1 at the mRNA (left panel) and protein (right panel) level.

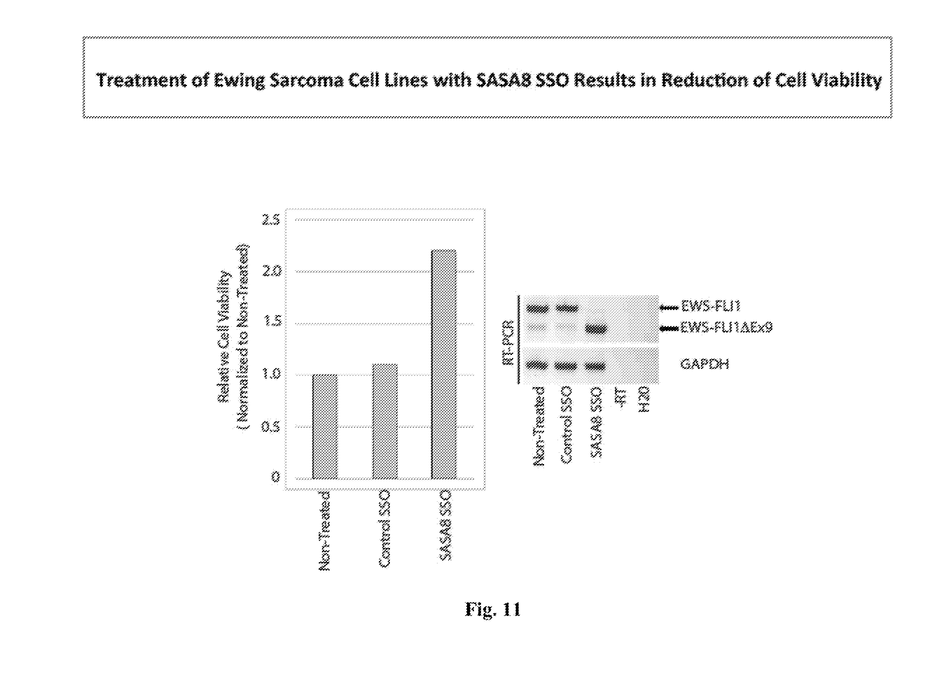

The left panel of FIG. 11 provides the results of experiments showing that treatment of Ewing Sarcoma cell lines with SASA8 SSO results in a reduction of cell viability. The right panel of FIG. 11 provides RT-PCR data confirming the generation of the truncated EWS-FLI1ΔEx9 mRNA in the cells treated with SASA8.

The present invention may also be defined in terms of the following Claims:

We claim:

1. An isolated oligonucleotide molecule comprising LSE9a (SEQ ID NO. 1).

2. An isolated oligonucleotide molecule consisting essentially of LSE9a (SEQ ID NO. 1).

3. An isolated oligonucleotide molecule consisting of LSE9a (SEQ ID NO. 1).

4. An isolated oligonucleotide molecule capable of hybridizing with the LSE9 target sequence SEQ ID NO. 3.

5. An isolated oligonucleotide molecule capable of hybridizing with the LSE9 target sequence SEQ ID NO. 3 under high stringency conditions.

6. An isolated oligonucleotide molecule that is substantially complementary to the LSE9 target sequence SEQ ID NO. 3.

7. An isolated oligonucleotide molecule according to any of the preceding claims, wherein the oligonucleotide comprises one or more naturally-occurring nucleotides.

8. An isolated oligonucleotide molecule according to any of claims 1-6, wherein the oligonucleotide comprises one or more non-naturally occurring man-made modified nucleotides.

9. An isolated oligonucleotide molecule according to any of claims 1-6, wherein every nucleotide in the oligonucleotide molecule is a non-naturally occurring man-made modified nucleotide.

10. A composition comprising an isolated oligonucleotide molecule according to any of the preceding claims.

11. The composition of claim 10, wherein the composition is a pharmaceutical composition.

12. A method of treating DSRCT in a subject in need thereof, the method comprising administering to the subject an effective amount of an isolated oligonucleotide molecule or composition according to claim 1.

13. The method of claim 12, wherein the subject has a EWS-WT1 chromosomal translocation.

14. The method of claim 12, wherein the subject has an EWS-WT1 driven DSRCT.

15. The method of claim 12, wherein the subject is a human.

16. A method of inducing apoptosis in DSRCT cells, the method comprising contacting DSRCT cells with an isolated oligonucleotide molecule or composition according to claim 1.

17. A method of inhibiting the growth of DSRCT cells, the method comprising contacting DSRCT cells with an isolated oligonucleotide molecule or composition according to claim 1.

18. A method of inhibiting the transcriptional activity of an oncogenic EWS-WT1 chimeric transcription factor in DSRCT cells, the method comprising contacting DSRCT cells with an isolated oligonucleotide molecule or composition according to claim 1.

19. An isolated oligonucleotide molecule comprising SASA8 (SEQ ID NO. 2).

20. An isolated oligonucleotide molecule consisting essentially of SASA8 (SEQ ID NO. 2).

21. An isolated oligonucleotide molecule consisting of SASA8 (SEQ ID NO. 2).

22. An isolated oligonucleotide molecule capable of hybridizing with the SASA8 target sequence SEQ ID NO. 4.

23. An isolated oligonucleotide molecule capable of hybridizing with the SASA8 target sequence SEQ ID NO. 4 under high stringency conditions.

24. An isolated oligonucleotide molecule that is substantially complementary to the SASA8 target sequence SEQ ID NO. 3.

25. An isolated oligonucleotide molecule according to any of claims 19-24, wherein the oligonucleotide comprises one or more naturally-occurring nucleotides.

26. An isolated oligonucleotide molecule according to any of claims 19-24, wherein the oligonucleotide comprises one or more man-made modified nucleotides.

27. An isolated oligonucleotide molecule according to any of claims 19-24, wherein every nucleotide in the oligonucleotide molecule is a non-naturally occurring man-made modified nucleotide.

28. A composition comprising an isolated oligonucleotide molecule according to any of claims 19-27.

29. The composition of claim 28, wherein the composition is a pharmaceutical composition.

30. A method of treating Ewing Sarcoma in a subject in need thereof, the method comprising administering to the subject an effective amount of an isolated oligonucleotide molecule or composition according to claim 19.

31. The method of claim 30, wherein the subject has a EWS-FLI1 chromosomal translocation.

32. The method of claim 30, wherein the subject has an EWS-FLI1 driven Ewing Sarcoma.

33. The method of claim 30, wherein the subject is a human.

34. A method of inducing apoptosis in Ewing Sarcoma cells, the method comprising contacting DSRCT cells with an isolated oligonucleotide molecule or composition according to claim 19.

35. A method of inhibiting the growth of Ewing Sarcoma cells, the method comprising contacting Ewing Sarcoma cells with an isolated oligonucleotide molecule or composition according to claim 19.

36. A method of inhibiting the transcriptional activity of an oncogenic EWS-FLI1 chimeric transcription factor in Ewing Sarcoma cells, the method comprising contacting Ewing Sarcoma cells with an isolated oligonucleotide molecule or composition according to claim 19.


SEQUENCE LISTING (4 sequences)

SEQ ID NO: 1 - DNA, 25 bp, Artificial Sequence
  Description of Artificial Sequence Synthetic oligonucleotide
agtttacgcacttgttttacctgta

SEQ ID NO: 2 - DNA, 25 bp, Artificial Sequence
  Description of Artificial Sequence Synthetic oligonucleotide
caggccaaggtaaactcaccagggt

SEQ ID NO: 3 - DNA, 25 bp, Artificial Sequence
  Description of Artificial Sequence Synthetic oligonucleotide
tacaggtaaaacaagtgcgtaaact

SEQ ID NO: 4 - DNA, 25 bp, Artificial Sequence
  Description of Artificial Sequence Synthetic oligonucleotide
accctggtgagtttaccttggcctg